(19) United States
(12) Patent Application Publication
(10) Pub. No.: US 2021/0244849 A1
(43) Pub. Date: Aug. 12, 2021

(54) L-PRF MESH REPAIR FOR INGUINAL HERNIA

(71) Applicants: James R. Strole, Scottsdale, AZ (US); Thomas Richard Swift, London (GB)

(72) Inventor: Valerio Di Nicola, Worthing (GB)

(73) Assignees: James R. Strole, Scottsdale, AZ (US); Thomas Richard Swift, London (GB)

(21) Appl. No.: 17/169,992

(22) Filed: Feb. 8, 2021

Related U.S. Application Data
(60) Provisional application No. 62/972,858, filed on Feb. 11, 2020.

(51) Int. Cl. A61L 27/22; A61L 27/36; A61L 27/58; A61L 27/56

(52) U.S. Cl. CPC A61L 27/225; A61L 27/3616; A61L 27/56; A61L 27/3691; A61L 27/58; A61L 27/3641

(57) ABSTRACT

A platelet-rich fibrin (PRF) three-dimensional, adhesive, biocompatible and biodegradable scaffold and/or membrane is obtained by collecting a blood sample, followed by centrifugation of the blood sample to obtain a platelet-rich fibrin clot and an exudate followed by compressing platelet-rich fibrin clot to extract the exudate until the final product being a platelet-rich fibrin three-dimensional, adhesive, biocompatible and biodegradable scaffold is obtained. A hyper-acute serum is obtained by collecting a blood sample, followed by centrifugation of said blood sample to obtain a platelet-rich fibrin clot and an exudate followed by compressing platelet-rich fibrin clot to obtain the membrane and extract the exudate being the final product hyper-acute serum. A process for the preparation of a platelet-rich fibrin three-dimensional, adhesive, biocompatible and biodegradable scaffold and/or membrane and a hyper-acute serum is also provided. A method for the treatment of inguinal hernia in a patient in need thereof is also provided.

### CROSS REFERENCE TO RELATED APPLICATIONS

This application claims the benefit of U.S. Provisional Application No. 62/972,858, filed Feb. 11, 2020, which is incorporated by reference as if fully set forth.

### FIELD OF THE INVENTION

The present invention relates to the field of inguinal hernia repair.

### BACKGROUND

Inguinal hernia repair is one of the most frequently performed operation in general surgery. Over 20 million hernia repair procedures per year are performed worldwide. Complications such as chronic inguinal pain (12%) and recurrence rate (11%) significantly impact the surgical results. The 4 main impacting factors affecting hernia repair results are: mesh material and design; mesh fixation; tissue healing and regeneration and the surgical technique. This analysis is followed by an explanation of how a new procedure, L-PRF-Open Mesh Repair, can improve both short- and long-term results.

Open inguinal hernia repair is the most common hernia repair technique and is performed by making an incision in the inguinal region by way of the subcutaneous tissue and the anterior fascia, and then the sac is identified and dissected from the cord/round ligament. Once the hernia sac is divided from the adjacent structures and examined for contents, this is pushed back into the peritoneal cavity, and finally, hernia repair is carried out.

Open inguinal hernia repair can be executed in two ways: primary repair such as Bassini or Shouldice techniques involves the sewing of the abdominal wall layers using sutures. This method is considered obsolete and has, in the last decades, been replaced by patch or mesh repair. Mesh repair relies on the placement of a mesh to seal the hernia defect and reinforce the surrounding tissue. The Lichtenstein technique requires stitches to fix the located mesh whereas other procedures fix it using either fibrin or cyanoacrylate glue.

In 2002, the European Union Hernia Trialists Collaboration, a group of surgical trialists who have participated in randomized trials of open mesh or laparoscopic groin hernia repair, analyzed 58 randomized controlled trials and concluded that the use of surgical meshes was superior to any other techniques [EU Hernia Trialists Collaboration Repair of groin hernia with synthetic mesh: Meta-analysis of randomized. Ann. Surg. 2002; 235:322-332.].

The new International Guidelines of the HerniaSurge Group recommend only the open Lichtenstein mesh technique and the laparoscopic mesh procedures TEP and TAPP as repair techniques in inguinal hernia surgery. Different meta-analyses comparing TEP and TAPP with the Lichtenstein technique could not deliver sufficient evidence to determine the greater effectiveness of one over the other technique [Kockerling F, Simons M. P. Current Concepts of Inguinal Hernia Repair. Visc Med 2018; 34:145-150; Scheuermann U, Niebisch S, Lyros O, Jansen-Winkeln B, Gockel I. Transabdominal preperitoneal (TAPP) versus Lichtenstein operation for primary inguinal hernia—a systematic review and meta-analysis of randomized controlled trials. BMC Surg 2017; 17: 55].

The results of hernia surgery are mainly measured by postoperative pain, time to return to work, short-term complications, chronic groin pain, and hernia recurrence.

The expected rate of recurrence following inguinal hernia repair is currently 11%.

Only 57% of all inguinal hernia recurrences occurred within 10 years after the hernia operation. Some of the remaining 43% of all recurrences happened only much later, even after more than 50 years [Kockerling F, Koch A, Lorenz R, Schug-Pass C, Stechemesser B, Reinpold W. How long do we need to follow-up our hernia patients to find the real recurrence rate? Front Surg 2015; 2:24.].

A further complication after inguinal hernia repair is chronic groin pain lasting more than 3 months, occurring in 10-12% of all patients. Approximately 1-6% of patients have severe chronic pain with long-term disability, thus requiring treatment [HerniaSurge Group: International guidelines for groin hernia management. Hernia 2018; 22:1-165.; Tebala G D, Tognoni V, Tristram Z, Macciocchi F, Innocenti P. Cyanoacrylate glue versus suture fixation of mesh in inguinal hernia open repair a randomized controlled clinical trial. Gastroenterol Hepatol. 2015; 2(5).]

In recent decades researchers have investigated various materials to optimize this aspect of mesh repair surgery and improve results. The fundamental material characteristics should be biocompatibility, resistance to infection, maintenance of adequate long-term tensile strength, rapid incorporation into the host tissue, sufficient flexibility and non-carcinogenic response.

Currently, most of the surgical meshes are chemically and physically biocompatible, non-toxic, and non-immunogenic but none of them is biologically inactive and this may variably impact the mesh suitability in the host tissue and the effective process of mesh integration [Di Nicola V, Regenerative Surgery in Open Mesh Repair for Inguinal Hernia. J Regen Med 2020, 9:1.157. DOI: 10.37532].

The mesh integration process has four main stages. The first biological response of the injured site starts with the coagulation of proteins around the prosthetic implant. Platelets adhere to these proteins activating the classical and alternative complement pathways, especially generating factors C3a and C5a that convey polymorphonucleocytes or neutrophils (PMNs), fibroblasts, smooth muscle cells and macrophages to the wound area in an ordered sequence. During this first stage or acute phase of inflammation, migratory PMNs phagocyte microorganisms and necrotic material. When exhausted PMNs die and release their cytoplasmic and granular contents near the mesh, this may cause an additional inflammatory response.

If the acute inflammatory response fails to eliminate the cause of injury and to restore injured tissue to normal physiology, this condition could develop into a state of chronic inflammation.

In the second stage, monocytes that migrated from the blood stream to the wound site differentiate into macrophages. In addition to macrophages, other primary cellular components such as plasma cells and lymphocytes actively contribute to the advanced inflammatory response. Macrophages start the phagocytosis of dead cells, necrotic tissue and consume foreign bodies and generally prepare the way for fibroblasts settlement [Tang L, Ugarova T. P, Plow E. F, Eaton J. W. Molecular determinants of acute inflammatory response to biomaterials. J. Clin. Invest. 1996; 97:1329-13234.].

In the third stage, in response to the presence of large indigestible foreign bodies, macrophages fuse into a foreign body giant cell in the attempt to seal the extraneous material in an epithelioid granuloma [Anderson J. M, Rodriguez A, Chang D. T. Foreign Body Reaction to Biomaterials. Semin. Immunol. 2008; 20:86-100].

The last stage is characterized by the replacement of damaged tissue with fibroblasts and other cell lineages; these produce the extracellular matrix (EMC) and generate the scar. Wound healing and scar formation can be affected by persistent inflammation and the severity of the primary injury [Di Nicola V, Regenerative Surgery in Open Mesh Repair for Inguinal Hernia. J Regen Med 2020, 9:1.157. DOI: 10.37532].

Fibroblasts are the cells that mediate the wound healing progression. These cells enter the wound site two to five days after the surgery, typically once the acute inflammatory response has receded. Fibroblasts proliferate at the wound site, reaching peak levels after one to two weeks. The main function of fibroblasts is to synthesize extracellular matrix (EMC) with collagen to regenerate the connective tissue. The EMC is itself involved in the regulation of inflammation, angiogenesis and connective tissue regeneration through the collagen matrix laid down by fibroblasts together with local GFs [Anderson J. M. Biological Response to Materials. Annu. Rev. Mater. Res. 2001; 31:81-110].

The optimum amount (density) of fibroblasts needed for the successful mesh integration is achieved approximately two weeks after surgery. Collagen is the principal biomechanical component of connective tissue, it provides strength and acts as a scaffold in the forms of type I, II and III. A normal fibroblastic biological response leads to the optimal synthesis of the connective tissue. Primarily immature, frail collagen type III is synthesized and excreted by fibroblasts as a monomeric form into the extracellular space where it polymerizes into an insoluble helical structure. A fragile collagen network is produced for around the first 21 days, and then there is a modification in the ratio of collagen type III and I. The collagen type III reduces and the type I, stronger and stable, arises. The mechanical strength increases progressively until 6 months after surgery [Elango S, Perumalsamy S, Ramachandran K, Vadodaria K. Mesh materials and hernia repair. Biomedicine (Taipei). 2017 September; 7 (3): 16.].

Therefore, the quality of connective tissue is significantly influenced by the collagen ratio type I/III and its contribution to making up the ECM. An altered Type I/III collagen ratio results in decreased tensile strength and mechanical stability. Thus, the alterations of collagen subtypes play a central role in the pathophysiology of hernia repair and mesh integration.

Recent Literature highlights the responsibility of enzymes like Matrix Metallo-Proteinases, MMPs and the lack of their inhibitors Tissue Inhibitors of Metalloproteinases, TIMPS to be the possible cause for the altered ratio of collagen subtypes. The main function of these enzymes is to degrade and expedite the turnover of the extracellular matrix (EMC) by acting on certain types of collagen and elastin. MMP-1 and MMP-13 are the principal matrix enzymes responsible for the type I, II, III collagen turn over. Therefore, the alterations in MMP-1 and MMP-13 protein expressions could have a role in the derangement of the ratio type I/III collagen. MMP-2 and MMP-9, derived from neutrophils, play an important role in degrading collagen types IV and V as well as elastin, fibronectin, and other EMC components. A direct correlation between the altered expression of MMP-2 and MMP-9 with the diminished level of TIMP-1 and TIMP-2 was observed in inguinal hernia patients [Rangaraj A, Harding K, Leaper D. Role of collagen in wound management. Wounds. 2011; 7(2): 54-63.].

Excessive fibroblasts and associated hyperactivity in the wound area will prolong the inflammatory phase resulting in increasing fibrosis. This will compromise the optimal synthesis of collagen and thereby the prosthesis integration. Optimizing conditions for the first two weeks post-surgery appears to be essential for the successful incorporation of the mesh and to produce healthy connective tissue. After 12 weeks the process is considered more stable and the overall strength of the scar increases gradually up to six months. The final result is a relatively less elastic tissue that has only 70-80% of the strength of the native connective tissue.

A non-absorbable prosthesis fully incorporated within newly formed collagen will help to optimize overall strength.

The fibrotic reaction generated by the body when a mesh is introduced is governed by the chemical nature of the material and its physical characteristics such as the filament structure and pore size.

Properties and design of the mesh influence the immune reaction, wound healing and integration process [Brown C. N., Finch J. G. Which mesh for hernia repair? Ann. R. Coll. Surg. Engl. 2010; 92:272-278.].

The “ideal” mesh should be suturable to be held in place, resistant to straining and loading under biaxial tension (e.g. defecation or lifting actions) especially during the first 3 months postoperative period. This prosthetic implant should promote a fast and organized integration process supporting tissue regeneration together with a minimal inflammatory reaction. There are over 70 meshes commercially available for hernia repair, each attempt to match the “ideal” mesh characteristics. They are classified according to the composition or type of material as first-generation (synthetic non-absorbable prosthesis), second-generation (mixed or composite prosthesis), and third-generation (biological prosthesis).

For inguinal hernia surgery, the most recommended are the macroporous, non-absorbable, monofilament, lightweight soft mesh. Polypropylene has been the material of choice and several companies produce in different shapes and at a range of price.

Mesh fixation is a controversial area in inguinal hernia surgery. The most common methods used to fix the hernia mesh are stitches, fibrin and cyanoacrylate glues. A minority of surgeons suggest for a non-fixation technique (mostly in TEP); however, reports about the rate of recurrence are incomplete and controversial.

The Lichtenstein technique is currently the most popular technique to repair unilateral primary groin hernias. In the Lichtenstein technique, the mesh fixation is provided by a Prolene (polypropylene) overrunning or single stitch suture to sew the mesh firmly to the surrounding structures: the pubic tubercle, inguinal ligament and muscles.

Some surgeons opt for absorbable stitch material such as polyglycolic acid (Vicryl) in the hope of reducing the risk of long-term nerve entrapment in the suture line.

Lichtenstein technique has a generally good reported outcome in Literature: easy to perform, low morbidity and good long-term results.

Nonetheless, several recent articles showed a high incidence of chronic inguinal pain, with an average incidence of 12%, and sometimes reported as high as 53%. Specifically, in the Lichtenstein operation, chronic groin pain can be due to nerve entrapment in the suture either in the scar tissue or neuroma development, inflammation of the periosteum of the pubic tubercle (traditionally taken into the first stitch), and persistent inflammation with foreign body over-reaction to the mesh [Erhan Y, Erhan E, Aydede H, Mercan M, Tok D Chronic pain after Lichtenstein and preperitoneal (posterior) hernia repair. Can J Surg 2008; 51(5): 383-387].

Many studies considered chronic postoperative pain as a surgical primary outcome but only a few of them evaluated the social impact of post-hernia repair chronic pain. This chronic groin pain has been reported to affect the social, sexual and work life of up to 6% of patients.

Lichtenstein technique has been always described as a “tension-free technique”. However, stitches do generate tension and stiffness in the inguinal area, particularly during muscle activity. This tension may cause postoperative mechanical pain, persistent inflammation and delay in recovery time.

To avoid the above problems and reduce the risk of chronic pain, different methods of mesh fixation have been considered and most of all tissue-compatible glues. The goal is to provide a suture-less fixation. The ideal adhesive material should be biocompatible, cheap and easy to store and use [Di Nicola V; Regenerative Surgery for Inguinal Hernia Repair. Clin Res Trials, 2020, 6: 1-8. doi: 10.15761/CRT.1000307]

The advantages of glue fixation are reduced postoperative acute and chronic pain (due to the nerve entrapment syndrome), improved hemostasis, and a faster operation.

There is no report in Literature of any difference in recurrence when comparing glue with stitch fixation.

The main components of Fibrin glues are concentrated fibrinogen, thrombin, and calcium chloride, thus duplicating the final stage of the coagulation cascade. Fibrin acts as a hemostatic barrier, adheres to the surrounding tissue, and operates as a scaffold for migrating fibroblasts. Fibrin glues are used as a tissue adhesive for a variety of surgical procedures. The main advantages of fibrin glues are tissue compatibility, biodegradability, and efficacy when applied to wet surfaces. Potential contamination by transmissible blood-borne pathogens has been criticized by some authors. Furthermore, Fibrin glue is expensive and difficult to store and to prepare for the surgical application. However, fibrin glue has given very good results in tension-free mesh fixation both in open and laparoscopic approaches [16-.Liu H, Zheng X, Gu Y, Guo S: A Meta-Analysis Examining the Use of Fibrin Glue Mesh Fixation versus Suture Mesh Fixation in Open Inguinal Hernia Repair. Dig Surg 2014; 31:444-451. doi: 10.1159/000370249]

Some surgeons consider the long-lateral-chain cyanoacrylates CAs the best choice for mesh fixation in open mesh repair for inguinal hernia. CAs is biocompatible, inexpensive and easy to store and use, it is an efficient way to seal the mesh to the nearby tissue and works as hemostatic although does not provide a scaffold or facilitate tissue regeneration.

The main problem of the CAs has been recognized in the general increase of macrophage response and local inflammation when compared to absorbable sutures and, it is also a permanent material with a long-term destiny that has not yet been clarified. [Pascual G, Mesa-Ciller C, Rodríguez M, Pérez-Köhler B, Gómez-Gil V, Fernández-Gutiérrez M et al. Pre-clinical assay of the tissue integration and mechanical adhesion of several types of cyanoacrylate adhesives in the fixation of lightweight polypropylene meshes for abdominal hernia repair. Plose one 2018; 13(11):e0206515.].

Platelet-rich fibrin (PRF) has been described as a second-generation autologous platelet concentrate (PRP) because it does not require any biochemical additives like anticoagulants or bovine thrombin for fibrin polymerization.

Autologous platelet concentrates are widely used as a bioactive surgical component to decrease inflammation and increase the speed of the healing process. In the last few years, several different techniques have been developed to produce platelet concentrates from blood.

Platelets play a key role as an autologous source of growth factors. Since their activation, platelets secrete multiple GFs including PDGF, TGF β-1, 2, and IGF-I [Naik B, Karunakar P, Jayadev M, Marshal V. R. Role of Platelet-rich fibrin in wound healing: A critical review. J. Conserv. Dent. JCD. 2013; 16:284-293].

However, PRF biological properties are rather different from PRP and cannot be considered a PRP development but rather a different biostimulator.

The preparation of PRF begins with the immediate centrifugation of the patient's venous blood collected in normal glass tubes.

PRF is an autologous biomaterial, made of a strong fibrin matrix that variably contains a high concentration of vital and non-vital: platelets, leucocytes and circulating MSCs; variable pool of cytokines an elevated concentration of long releasing growth factors (GFs). These include platelet-derived growth factor (PDGF A-B), vascular endothelial growth factor (VEGF), transforming growth factor (TGF β-1,2), insulin-like growth factor (IGF-I), epidermal growth factor (EGF); connective tissue growth factor (CTGF); bone morphogenetic protein 2 (BMP-2), an elevated concentration of fibrin, fibronectin, vitronectin, and thrombospondin, a variable pool of heat shock proteins HSPs. [Dhurat R, Sukesh M S. Principles and Methods of Preparation of Platelet-Rich Plasma: A Review and Author's Perspective. J Cutan Aesthet Surg. 2014 October-December; 7(4): 189-197].

For PRF production, blood samples are collected from the patient in sterile plastic-coated tubes (8-10 ml) to be immediately treated in a centrifuge, are known various protocols to centrifuge blood and thereby produce different kinds of PRF with different properties.

L-PRF typically is made with 9 ml fresh blood in glass-coated plastic tubes, immediately centrifuged at 2700 rpm for 12 minutes.

Centrifuge stability, vibration and the temperature developed in the tubes determine the properties and quality of the final PRF. Specifically, the centrifuge characteristics impact on the making of L-PRF clot and membrane and this, in turn, determines cells survival, fibrin architecture and also the GFs contained in them [Doan Ehrenfest D M, Pinto N R, Pereda A, Jimenez P, Corso M D, Kang B S et al. Impact of the centrifuge characteristics and centrifugation protocols on the cells, growth factors, and fibrin architecture of a leukocyte- and platelet-rich fibrin (L-PRF) clot and membrane. Platelets. 2018 March; 29 (2): 171-184].

L-PRF shows a strongly polymerized thick fibrin matrix and large numbers of cells appear alive with a normal shape, including activated lymphocytes [Madurantakam P, Yoganarasimha S, Hasan F K. Characterization of Leukocyte-platelet Rich Fibrin, A Novel Biomaterial. J Vis Exp. 2015; (103): 53221.].

L-PRF membranes in vitro maintain good health for 7 days and slowly release their growth factors for at least 7 days.

After centrifugation, through the activation of autologous thrombin, a fibrin clot is created. Three distinct layers can be seen in the tube as red blood corpuscles RBCs at the bottom of the tube, platelet-poor plasma PPP on the top of the tube, and the PRF clot in the middle of the tube.

The PRF clot can be removed from the tube with surgical tweezers. The clot by itself contains a great amount of exudate, which is rich in growth factors, and this exudate can be pressed out by gentle compression of the clot in order to obtain PRF membranes.

Serum squeezed out from the PRF clot, called hyper-acute serum, has a greater cell proliferative effect on different connective cell lineages such as bone marrow mesenchymal stem cells (MSCs), osteoblasts, chondroblasts and fibroblast cells [Simon M, Major B, Vácz G, Kuten O, Hornyák I, Hinsenkamp A et al. The Effects of Hyperacute Serum on the Elements of the Human Subchondral Bone Marrow Niche. Stem Cells Int. 2018; 2018:4854619]

After removing the hyper-acute serum fraction, the remaining PRF membrane is a three-dimensional, adhesive, biocompatible and biodegradable scaffold. The membrane surface and ECM structure facilitate contact and cell interactions. Furthermore, PRF membranes can slowly release bioactive molecules that facilitate migration, adhesion and proliferation of local MSCs [Di Liddo R, Bertalot T, Borean A, Pirola I, Argentoni A, Schrenk S et al. Leucocyte and Platelet-rich Fibrin: A carrier of autologous multipotent cells for regenerative medicine. J. Cell. Mol. Med. 2018; 22:1840-1854].

During centrifugation, the soluble fibrinogen contained in the plasma transforms to fibrin polymerizing to a three-dimensional structure. The activated platelets and some leukocytes are entrapped in the fibrin net. Consequently, a storage pool of growth factors is formed from platelets and leukocytes upon activation. Platelets can be observed both on the surface and inside of the PRF membranes. The platelets' GFs are widely used in soft and bone tissue regeneration; one of the better known is the PDGF, which enhances MSCs adhesion and proliferation.

PRF has been successfully used for the treatment of non-responding skin ulcers including diabetic foot ulcers (DFU), pressure ulcers (PU), acute surgical wounds, and venous leg ulcers (VLUs) [Pinto, N. R, Ubilla M, Zamora Y, Del Rio V, Dohan Ehrenfest D. M, Quirynen M. Leucocyte- and platelet-rich fibrin (L-PRF) as a regenerative medicine strategy for the treatment of refractory leg ulcers: a prospective cohort study. Platelets 2017; 29, 468-475.].

In dentistry and maxillofacial surgery, the application of PRF membrane is widespread. There are numerous described procedures including the treatment of periodontal bony defects and regeneration, ridge preservation, sinus-floor elevation, implant surgery, and the creation of the PRF bone block [Saluja H., Dehane V., Mahindra U. Platelet-Rich fibrin: A second generation platelet concentrate and a new friend of oral and maxillofacial surgeons. Ann. Maxillofac. Surg. 2011; 1:53-57].

In dermatology and plastic surgery have been reports of dermal fibroblasts migration and activation resulting in the increase of collagen synthesis of the skin exposed to PRF treatment [Desai C. B, Mahindra U. R, Kini Y. K, Bakshi M. K. Use of Platelet-Rich Fibrin over Skin Wounds: Modified Secondary Intention Healing. J. Cutan. Aesthet. Surg. 2013]

In 2009, De Hingh I. H. J. T et al, described the use of autologous P-RFS (Platelet-Rich Fibrin Sealant) on 22 patients, intending to fix the mesh replacing the human/bovine fibrin glue and declaring the hemostatic and antibacterial effects of P-RFS. The preparation required a large amount of patient's own blood (120 ml) stored into a designated preparation unit containing sodium citrate for anticoagulation. Afterwards, the container was placed in the centrifuge for 23 min to produce an average of 6 ml of P-RFS. With a spray applicator, the P-RFS was applied along the ligament and the medial and cranial side of the mesh [De Hingh I. H. J. T, Nienhuijs S. W, Overdevest E. P, Scheele K, Everts P. A. M. Mesh Fixation with Autologous Platelet-Rich Fibrin Sealant in Inguinal Hernia Repair. Eur Surg Res 2009; 43:306-309].

### Technical Problem

The inventors of the present invention investigated how to use L-PRF clot and its constituents L-PRF membranes and hyper-acute serum to fix the mesh in inguinal hernia open mesh repair procedure. The procedure aims 1) to fix the mesh with a product acting as a glue to achieve a free tension technique and therefore, avoiding stitches, tensions and tractions of tissue, 2) the L-PRF works as a scaffold for fibroblasts and local MSCs and, 3) promote tissue healing and regeneration.

The glue designed by the present inventors is less expensive than either fibrin glue or cyanoacrylate, provides similar scaffolding properties to fibrin glue, eliminates the risk of transmission of blood pathogens, has beneficial anti-inflammatory properties as opposed to the characteristic property and problem of cyanoacrylates that is the promotion of inflammatory tissue reactions.

Most importantly, L-PRF exhibits both good fixation capacity and strong tissue regeneration properties, minimizes the local inflammatory acute response, streamlining the integration of the mesh through rapid fibroblast colonization and efficient collagen production. These elements ultimately in combination result in a faster and more effective wound healing process.

The present inventors also designed a novel surgical method for the treatment of inguinal hernia.

There are four main impacting factors affecting hernia repair results, which are mesh material and network design; mesh fixation; tissue healing and regeneration and, surgical technique.

The Lichtenstein technique, known in the art, promotes a rigid fixation of the mesh, recommending non-absorbable overrunning stitches (Prolene) to sew the mesh to the inguinal ligament and surrounding muscles structures; this generates tension, inflammation and pain. In addition, the risk that a permanent suture would trap a nerve is reasonably high (average 11-12% in Literature) causing chronic inguinal pain, which quite often requires local infiltration or unreliable nerve-lysis operations.

The present inventors found that the above risks can be significantly reduced by fixing the mesh to the trasversalis (posterior) fascia with a platelet-rich fibrin (PRF) three-dimensional, adhesive, biocompatible and biodegradable scaffold and/or membrane and by performing a real “tension-free” surgery technique.

The platelet-rich fibrin three-dimensional, adhesive, biocompatible and biodegradable scaffold and/or membrane of the present invention, also named L-PRF, is different from P-RFS ad disclosed in De Hingh I. H. J. T et al.

In 2009, De Hingh I. H. J. T et al, described the use of autologous P-RFS (Platelet-Rich Fibrin Sealant) on 22 patients. The aim was fixing the mesh replacing the human/bovine fibrin glue. The advantages claimed focused on the P-RFS hemostatic and antibacterial effects. The preparation of the blood required a large amount of patient's own blood (120 ml) and stored into a designated preparation unit containing sodium citrate for anticoagulation. Afterwards, the container was placed in the centrifuge for 23 min to produce an average of 6 ml of P-RFS. With a spray applicator, the P-RFS was applied along the ligament and the medial and cranial side of the mesh.

In the preparation of L-PRF, the amount of necessary blood to produce L-PRF is greatly smaller than P-RFS, the blood tube used does for not contain any anticoagulation medication, such as sodium citrate used for P-RFS procedure, the centrifugation parameters are different (time, speed and type of centrifuge).

In the surgical method of the present invention, the way to apply the L-PRF is completely different because using membrane and hyper-acute serum while P-RFS produces a fluid applied through a spray applicator. P-RFS is used as glue and/or a sealant and is not suggested any tissue regeneration property or capability to promoting mesh integration acceleration.

L-PRF shows, satisfactory, reliable and simple fixation of the mesh; superior hemostasis, less local inflammation; avoidance of nerve entrapment; streamlining the mesh integration process and tissue regeneration.

The L-PRF is an autologous platelet-rich fibrin centrifuge product, wherein the centrifuge characteristics and centrifugation protocols impact significantly on the characteristics of PRF leading the production of L-PRF with good glue and scaffolding performance and tissue regeneration properties.

In particular, PRF combines the benefits of fibrin glue sealant with the ability to streamline the integration of the mesh by optimizing connective tissue regeneration. Moreover, PRF regenerative capacities substantially prevent chronic fibrotic inflammation, mesh retraction, hard and painful scars and chronic nerve entrapment syndrome.

The clinical perspective of the PRF-open mesh repair is the reduction of postoperative pain, to accelerate the patient's recovery, prevent the chronic inguinal pain, reduce the rate of recurrence and reduce financial costs of the operation.

Lichtenstein technique promotes a rigid fixation of the mesh, recommending non-absorbable overrunning stitches (Prolene) to sew the mesh to the inguinal ligament and surrounding muscles structures; this generates tension, inflammation and pain.

In addition, the risk that a permanent suture would trap a nerve is reasonably high (average 11-12% in Literature) causing chronic inguinal pain, which quite often requires local infiltration or unreliable nerve-lysis operations.

These risks are significantly reduced by fixing the mesh to the trasversalis (posterior) fascia with the L-PRF and making a real “tension-free” technique.

### SUMMARY

The present invention provides a PRF (platelet-rich fibrin) three-dimensional, adhesive, biocompatible and biodegradable scaffold and/or membrane obtained by collecting a blood sample, followed by centrifugation (any time and speed) of said blood sample to obtain a platelet-rich fibrin clot and an exudate followed by compressing platelet-rich fibrin clot to extract the exudate until the final product being a Platelet-rich fibrin three-dimensional, adhesive, biocompatible and biodegradable scaffold is obtained.

The invention also provides a hyper-acute serum obtained by collecting a blood sample, followed by centrifugation of said blood sample to obtain a platelet-rich fibrin clot and an exudate followed by compressing platelet-rich fibrin clot to obtain the membrane and extract the exudate being the final product hyper-acute serum.

It is another object of the present invention a process for the preparation of a Platelet-rich fibrin three-dimensional, adhesive, biocompatible and biodegradable scaffold and/or membrane and a hyper-acute serum comprising the following steps:

a) collecting blood samples, followed by centrifugation to obtain a platelet-rich fibrin clot and an exudate;
b) gently compressing the platelet-rich fibrin clot as obtained in step a) to extract the exudate to obtain the final products being the hyper-acute serum and a platelet-rich fibrin (PRF) three-dimensional, adhesive, biocompatible and biodegradable scaffold and/or membrane.

The present invention also provides membrane and hyper-acute serum obtained by collecting blood samples, followed by centrifugation, followed by compressing the Platelet-rich fibrin clot as obtained in step a) to extract an exudate of hyper-acute serum to obtain the hyper-acute serum as a final product.

It is another object of the present invention a method for the treatment of Inguinal Hernia in a patient in need thereof comprising the steps of:

a) performing a small transverse incision on the inguinal region;
b) positioning a self-retaining retractor with smooth non-traumatic branches;
c) sharply cutting tissues avoiding any stretching or shredding during the dissection;
d) preparation with minimal manipulation of the sac from the cord and to make the space where the mesh will be located, minimizing the detachment of tissues and respecting the nerves that cross the area, while hemostasis checked step by step;
e) isolation and examination of the content of the sac of step d);
f) repositioning of the sac into the abdomen and the internal inguinal ring refashioned using stitches wherein in case of direct hernia the trasversalis fascia is approximated with the same suture material;
g) customization of a mesh to the patient's inguinal region, wherein the mesh is a soft, light, macropores, monofilament, polypropylene mesh BARD, designed according to the shape and size of the inguinal canal and fixed in place with a 2 cm overlap of the mesh above the tubercle;
h) mesh fixation by using a Platelet-rich fibrin three-dimensional, adhesive, biocompatible and biodegradable scaffold and/or membrane and a hyper-acute serum to fix the mesh and secure a tension-free technique prepared by collecting patient's blood in glass-coated plastic tubes and immediately centrifuging o obtain three distinct layers in the centrifugation tube: red blood corpuscles RBCs at the bottom of the tube, platelet-poor plasma PPP on the top of the tube, and the PRF clot in the middle of the tube, removing the PRF clot was from the tube with surgical tweezers; squeezing out serum from the PRF clot to generates PRF membranes and the hyper-acute serum, PRF clots used to fix the mesh;
i) application of both components membranes and hyper-acute serum on the posterior fascia (trasversalis) and the mesh attached over them;
j) using two single stitches to adjust the mesh: one in the tubercle area avoiding the periosteum and another one to close the mesh tails;
k) suturing the anterior fascia below the spermatic cord using stitches to press the mesh between the anterior and the trasversalis fascia;
l) fat suturing and skin intradermic suture.

### BRIEF DESCRIPTION OF THE DRAWINGS

The following detailed description of the preferred embodiment of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1 is a depiction of a patient's blood in a glass-coated plastic tube immediately centrifuging to obtain three distinct layers in the centrifugation tube;

FIG. 2 is shows a PRF clot being removed from a centrifuge tube;

FIG. 3 shows a PRF and hyper-acute serum positioned onto the transversalis fascia;

FIG. 4 shows a PRF being placed in position; and

FIG. 5 shows PRF applied to the posterior wall of the inguinal canal.

### DETAILED DESCRIPTION OF THE PREFERRED EMBODIMENTS

Certain terminology is used in the following description for convenience only and is not limiting. The words “right,” “left,” “top,” and “bottom” designate directions in the drawings to which reference is made. The words “a” and “one,” as used in the claims and in the corresponding portions of the specification, are defined as including one or more of the referenced items unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

Within the meaning of the present invention PRF is an autologous biomaterial that is made of a strong fibrin matrix which variably contains a high concentration of vital and non-vital: platelets, leucocytes and circulating MSCs; variable pool of cytokines an elevated concentration of long releasing growth factors (GFs). These include platelet-derived growth factor (PDGF A-B), vascular endothelial growth factor (VEGF), transforming growth factor (TGF β-1,2), insulin-like growth factor (IGF-I), epidermal growth factor (EGF); connective tissue growth factor (CTGF); bone morphogenetic protein 2 (BMP-2), an elevated concentration of fibrin, fibronectin, vitronectin, and thrombospondin, a variable pool of heat shock proteins HSPs. [Dhurat R, Sukesh M S. Principles and Methods of Preparation of Platelet-Rich Plasma: A Review and Author's Perspective. J Cutan Aesthet Surg. 2014 October-December; 7(4): 189-197].

For PRF production, blood samples are collected from the patient in sterile plastic-coated tubes to be immediately treated in a centrifuge, are known various protocols to centrifuge blood and thereby produce different kinds of PRF with different properties.

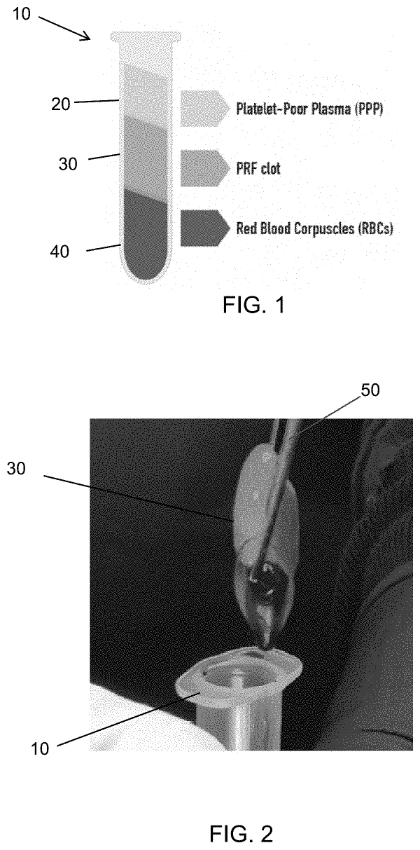

After centrifugation, through the activation of autologous thrombin, a fibrin clot is created. As depicted in FIG. 1, three distinct layers can be seen in the tube 10 as red blood corpuscles RBCs 40 at the bottom of the tube, platelet-poor plasma PPP 20 on the top of the tube, and the PRF clot 30 in the middle of the tube.

The PRF clot 30 can be removed from the tube 10 with surgical tweezers 50 (FIG. 2). The clot 30 by itself contains a great amount of exudate, which is rich in growth factors, and this exudate can be pressed out by gentle compression of the clot in order to obtain PRF membranes.

Serum squeezed out from the PRF clot, called hyper-acute serum, has a greater cell proliferative effect on different connective cell lineages such as bone marrow mesenchymal stem cells (MSCs), osteoblasts, chondroblasts and fibroblast cells [Simon M, Major B, Vácz G, Kuten O, Hornyák I, Hinsenkamp A et al. The Effects of Hyperacute Serum on the Elements of the Human Subchondral Bone Marrow Niche. Stem Cells Int. 2018; 2018:4854619].

PRF does not require any biochemical additives like anticoagulants or bovine thrombin for fibrin polymerization to be made

PRF can be produced using different amount of blood generally 8-12 ml per vials (red coated tube) and can be produced in multiple vials up to the maximum amount that can be allocated in the centrifuge.

Therefore, other PRFs produced with different amounts of blood, centrifuge speed and time and, with the characteristics reported above and below should be considered suitable for the claimed invention.

A PRF (platelet-rich fibrin) three-dimensional, adhesive, biocompatible and biodegradable scaffold and/or membrane is obtained by collecting a blood sample, followed by centrifugation of said blood sample to obtain a platelet-rich fibrin clot 30 and an exudate followed by compressing platelet-rich fibrin clot to extract the exudate until the final product being a platelet-rich fibrin three-dimensional, adhesive, biocompatible and biodegradable scaffold is obtained.

A hyper-acute serum is obtained by collecting a blood sample, followed by centrifugation of said blood sample to obtain a platelet-rich fibrin clot and an exudate followed by compressing platelet-rich fibrin clot to obtain the membrane and extract the exudate being the final product hyper-acute serum.

A process for the preparation of a Platelet-rich fibrin three-dimensional, adhesive, biocompatible and biodegradable scaffold and/or membrane and a hyper-acute serum includes the following steps:

a) collecting blood samples, followed by centrifugation to obtain a platelet-rich fibrin clot and an exudate;
b) gently compressing the platelet-rich fibrin clot as obtained in step a) to extract the exudate to obtain the final products being the hyper-acute serum and a platelet-rich fibrin (PRF) three-dimensional, adhesive, biocompatible and biodegradable scaffold and/or membrane.

A method for the treatment of inguinal hernia in a patient in need thereof includes the steps of:

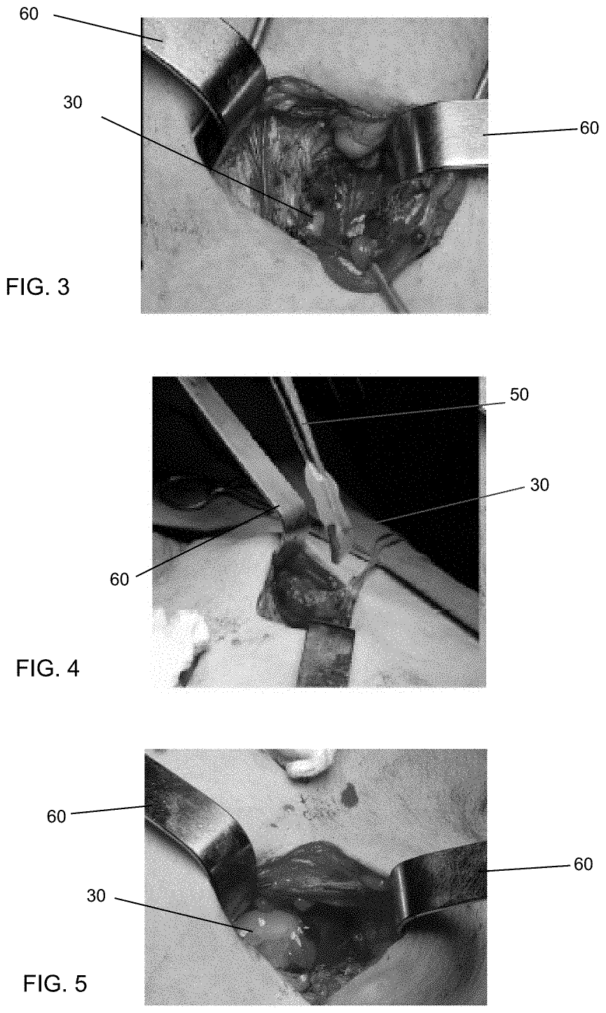

a) performing a small transverse incision on the inguinal region;
b) positioning a self-retaining retractor 60 with smooth non-traumatic branches;
c) sharply cutting tissues avoiding any stretching or shredding during the dissection;
d) preparation with minimal manipulation of the sac from the cord and to make the space where the mesh will be located, minimizing the detachment of tissues and respecting the nerves that cross the area, while hemostasis checked step by step;
e) isolation and examination of the content of the sac of step d);
f) repositioning of the sac into the abdomen and the internal inguinal ring refashioned using stitches wherein in case of direct hernia the trasversalis fascia is approximated with the same suture material;
g) customization of a mesh to the patient's inguinal region, wherein the mesh is a soft, light, macropores, monofilament, polypropylene mesh BARD, designed according to the shape and size of the inguinal canal and fixed in place with a 2 cm overlap of the mesh above the tubercle;
h) mesh fixation by using a platelet-rich fibrin three-dimensional, adhesive, biocompatible and biodegradable scaffold and/or membrane and a hyper-acute serum to fix the mesh and secure a tension-free technique prepared by collecting patient's blood in glass-coated plastic tubes and immediately centrifuging to obtain three distinct layers in the centrifugation tube: red blood corpuscles RBCs at the bottom of the tube, platelet-poor plasma PPP on the top of the tube, and the PRF clot in the middle of the tube, removing the PRF clot was from the tube with surgical tweezers; squeezing out serum from the PRF clot to generates PRF membranes and the hyper-acute serum, PRF clots used to fix the mesh;
i) application of both components membranes and hyper-acute serum on the posterior fascia (trasversalis) and the mesh attached over them (FIG. 3);
j) using two single stitches to adjust the mesh: one in the tubercle area avoiding the periosteum and another one to close the mesh tails;
k) suturing the anterior fascia below the spermatic cord using stitches to press the mesh between the anterior and the trasversalis fascia; and
l) fat suturing and skin intradermic suture.

The centrifugation of blood sample is performed at any appropriate and/or effective and/or suitable combination of time and speed to obtain the final products, i.e. a platelet-rich fibrin three-dimensional, adhesive, biocompatible and biodegradable scaffold and/or a hyper-acute serum. An example of an appropriate combination is centrifugation at 2700 rpm for 12 minutes.

In an embodiment the platelet-rich fibrin (PRF) three-dimensional, adhesive, biocompatible and biodegradable scaffold and/or membrane is obtained by collecting blood samples, followed by centrifugation, wherein the blood sample is 9 ml of fresh blood centrifuged at 2700 rpm for 12 minutes or 10 ml of fresh blood centrifuged at 1500 rpm for 14 minutes. Preferably the hyper-acute serum is obtained by collecting blood samples, followed by centrifugation, wherein the blood sample is 9 ml of fresh blood centrifuged at 2700 rpm for 12 minutes or 10 ml of fresh blood centrifuged at 1500 rpm for 14 minutes.

In an embodiment the process for the production of a platelet-rich fibrin (PRF) three-dimensional, adhesive, biocompatible and biodegradable scaffold and/or membrane and a hyper-acute serum comprises the following steps:

a) collecting blood samples, followed by centrifugation, preferably the blood sample is 9 ml of fresh blood which is immediately centrifuged at 2700 rpm for 12 minutes or the blood sample is 10 ml of fresh blood immediately centrifuged at 1500 rpm for 14 minutes; and
b) gently compressing the platelet-rich fibrin (PRF) clot as obtained in step a) to extract the exudate to obtain the final product being the hyper-acute serum to obtain a Platelet-rich fibrin (PRF) three-dimensional, adhesive, biocompatible and biodegradable scaffold and/or membrane and a hyper-acute serum as final products.

The surgical method includes any variation such as laparoscopy, including TAAP and TEP, and different types of suture material.

Preferably in step h), 3 to 5 L-PRF clots are used to fix the mesh.

Stitches can be polyglactin (vicryl) 2-0 or polyglycolic acid-Vicryl.

The open mesh repair procedure can be delivered under local or general anesthesia.

Preferably the platelet-rich fibrin (PRF) is L-PRF.

In an embodiment the treatment of an inguinal hernia comprises the steps of:

a) performing a small transverse incision of 5-6 cm on the inguinal region;
b) positioning a self-retaining retractor with smooth non-traumatic branches;
c) sharply cutting tissues avoiding any stretching or shredding during the dissection;
d) preparation with minimal manipulation of the inguinal sac from the cord and to make the space where the mesh will be located, minimizing the detachment of tissues and respecting the nerves that cross the area, while hemostasis checked step by step;
e) isolation and examination of the content of the sac of step d);
f) repositioning of the inguinal sac into the abdomen and the internal inguinal ring refashioned using polyglactin (vicryl) 2-0 or polyglycolic acid-Vicryl) stitches wherein in case of direct hernia the trasversalis fascia is approximated with the same suture material;
g) customization of a mesh to the patient's inguinal region, wherein the mesh is a soft, light, macroporous, monofilament, polypropylene mesh BARD, designed according to the shape and size of the inguinal canal and fixed in place with a 2 cm overlap of the mesh above the tubercle;
h) mesh fixation by using a Platelet-rich fibrin (PRF) three-dimensional, adhesive, biocompatible and biodegradable scaffold and/or membrane and a hyper-acute serum to fix the mesh and secure a tension-free technique prepared by collecting 9 ml fresh patient's blood in glass-coated plastic tubes and immediately centrifuging at 2700 rpm for 12 minutes to obtain three distinct layers in the centrifugation tube: red blood corpuscles RBCs at the bottom of the tube, platelet-poor plasma PPP on the top of the tube, and the PRF clot in the middle of the tube, removing the L-PRF clot was from the tube with surgical tweezers; squeezing out serum from the PRF clot to generates PRF membranes and the hyper-acute serum, at least 3 L-PRF clots used to fix the mesh;
i) application of both components membranes and hyper-acute serum on the posterior fascia (trasversalis) and the mesh attached over them;
j) using two single stitches in Vicryl 2-0 or polyglycolic acid-Vicryl to adjust the mesh: one in the tubercle area avoiding the periosteum and another one to close the mesh tails;
k) suturing the Anterior fascia below the spermatic cord using Vicryl 2-0 or polyglycolic acid-Vicryl stitches to press the mesh between the anterior and the trasversalis fascia; and
l) fat suturing with Vicryl 2-0 or polyglycolic acid-Vicryl and skin intradermic suture with Monocryl 3-0.

In an embodiment a small (5-6 cm) transverse incision on the inguinal region reduces skin tension following the natural Langer's lines; they are linear clefts in the skin that indicate the direction and orientation of the underlying collagen fibers. A small incision provides a proper exposition of the inguinal region. Retractors need to be moved gently; they should have smooth and not dentate edges.

Minimal manipulation is suggested to avoid any stretching and shredding of tissue. Tissue needs to be sharply cut following a constant dissection line, recognizing any different tissue layers to perform those minimal dissections necessary for completing the operation. Hemostasis needs to be checked step by step.

After the inguinal sac has been isolated and its contents examined, it will be repositioned into the abdomen and the internal inguinal ring refashioned using polyglactin (vicryl) 2-0 stitches. In the case of direct hernia, the trasversalis fascia needs to be approximated with the same suture material.

In preparing a suitable space to position the mesh, it is important to minimize the detachment of tissues and in particular to respect as much as possible the nerves that cross the area. The choice of an appropriate size, soft, light, macroporous, monofilament, polypropylene mesh is fundamental, as is the fixation method.

L-PRF clot with both components membrane and hyper-acute serum was used to fixing the mesh and secure a real tension-free technique.

Only two single stitches have been applied to the mesh: one in the tubercle area and another one to close the mesh tails. Vicryl 2-0 absorbable is always used to reduce risks of chronic nerve entrapment syndrome.

The anterior fascia is sutured below the spermatic cord using vicryl 2-0 stitches, to reduce the empty space and press the mesh between the trasversalis (posterior) and anterior fascia, ensuring that the spermatic cord is not on the way.

The fascia, sutured with absorbable (vicryl 2-0) stitches, places and compresses the mesh in the right space reducing the postoperative collection, indeed facilitating the integration process.

These basic principles reduce edema, collection and postoperative acute inflammation. These are the main factors that hinder the integration process and trigger postoperative pain.

In particular, PRF combines the benefits of fibrin glue sealant with the ability to streamline the integration of the mesh by optimizing connective tissue regeneration. Moreover, PRF regenerative capacities substantially prevent chronic fibrotic inflammation, mesh retraction, hard and painful scars and chronic nerve entrapment syndrome.

L-PRF shows some additional and essential clinical improvements: satisfactory fixation of the mesh; superior hemostasis; less local inflammation; avoidance of nerve entrapment; streamlining the mesh integration process and tissue regeneration.

The L-PRF is an autologous platelet-rich fibrin centrifuge product. The centrifuge characteristics and centrifugation protocols impact significantly on the characteristics of PRF.

The centrifugation protocol we have been using enables the production of L-PRF with good glue and scaffolding performance and also with tissue regeneration properties.

The surgical method, also named PRF-open mesh repair, is a tension-free technique that follows sound regenerative surgery principles.

The surgical technique, material choice, biological method of fixation and the regenerative properties of PRF, all described above, minimize wound site inflammation and assist correct integration of the mesh.

Postoperative advantages: less pain and shorter time of recovery are clear short-term benefits. Mid-long term benefits: lower incidence of chronic pain due to a correct process of wound healing and minimizing the risk of nerve entrapment.

Clinical Results

5 male patients were treated with the PRF-open mesh repair technique. Patients' average age was 52; 4 patients affected by single primary inguinal hernia and 1 had a bilateral one. 3 direct and 3 indirect inguinal hernias were intraoperatively detected. 3 patients were ASA II, 1 ASA I and 1 ASA III.

Four patients had general anesthesia and one had local. A small (5-6 cm) transverse incision in the inguinal region was performed. A self-retaining retractor with smooth non-traumatic branches positioned. Tissues were sharply cut avoiding any stretching or shredding during the dissection. Minimal manipulation used to prepare the sac from the cord and to make the space where the mesh will be located. It is important to minimize the detachment of tissues and in particular to respect as much as possible the nerves that cross the area. hemostasis checked step by step.

After the inguinal sac was isolated and content examined, it was repositioned into the abdomen and the internal inguinal ring refashioned using polyglactin (vicryl) 2-0 stitches. In case of direct hernia, the trasversalis fascia was approximated with the same suture material.

The mesh was then customized to be suitable for the patient's inguinal region. We choose a soft, light, macroporous, monofilament, polypropylene mesh BARD.

The mesh was designed according to the shape and size of the inguinal canal and fixed in place with a 2 cm overlap of the mesh above the tubercle.

Mesh fixation. We used L-PRF clot (Leukocyte-Platelet Rich Fibrin) with both components: membrane and hyper-acute serum to fix the mesh and secure a tension-free technique.

The L-PRF clot was prepared with an IntraSpin centrifuge using 9 ml fresh patient's blood in glass-coated plastic tubes and immediately centrifuged at 2700 rpm for 12 minutes.

After centrifugation, three distinct layers were in the tube: red blood corpuscles RBCs at the bottom of the tube, platelet-poor plasma PPP on the top of the tube, and the PRF clot in the middle of the tube.

The L-PRF clot was removed from the tube with surgical tweezers. Serum squeezed out from the PRF clot generates PRF membranes and the hyper-acute serum. We have used 3-5 L-PRF clots to fix the mesh. Both components membranes and hyper-acute serum were applied on the posterior fascia (trasversalis) and the mesh attached over them.

Only two single stitches in Vicryl 2-0 used to adjust the mesh: one in the tubercle area avoiding the periosteum and another one to close the mesh tails.

Anterior fascia closure. Fascia sutured below the spermatic cord using Vicryl 2-0 stitches to press the mesh between the anterior and the trasversalis fascia.

Fat sutured with Vicryl 2-0 and skin intradermic suture with monocryl 3-0.

Buvicaine 20 ml 0.5% as local anesthetic infiltration at the end of the procedure.

TABLE 1
Has been reported the intensity of pain (VAS) at 3 hours, 24 hours, 48 hours, 7
days, 15 days, 1 month, 3 months and 6 months after the operation

| | 3 h | 24 h | 48 h | 7 days | 15 days | 1 months | 3 months | 6 months |
|---|---|---|---|---|---|---|---|---|
| Right | 1 | 2 | 1 | 0 | 0 | 0 | 0 | 0 |
| Hernia | | | | | | | | |
| Left | 1 | 6 | 5 | 4 | 4 | 2 | 0 | 0 |
| Hernia | | | | | | | | |

Post-operative VAS score reported during follow up for the Right (PRF-opens mesh repair) and the Left inguinal hernia (Lichtenstein technique).

Oral paracetamol was prescribed after discharge 1 gr TDS per 5 days. Postoperative pain was measured with VAS (Visual Analog Scale) by direct interview or by a phone call at 3 hours, 24 hours, 48 hours, 7 days, 15 days, 1 month, 3 months and 6 months after the operation.

Clinical checks were set at 1 month and 6 months. Chronic groin pain was diagnosed whether still present at the 6th-month follow-up. Data were anonymized and stored and elaborated in an electronic database. As highlighted in Table 1 above, post-operative pain in the PRF-open mesh repair was not reported at 7 days whereas in the Lichtenstein technique, post-operative pain was reported up to one month.

The references cited throughout this application are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

1. A platelet-rich fibrin (PRF) three-dimensional, adhesive, biocompatible and biodegradable scaffold and/or membrane obtained by collecting a blood sample, followed by centrifugation of said blood sample to obtain a platelet-rich fibrin clot and an exudate followed by compressing platelet-rich fibrin clot to extract the exudate until the final product, being a platelet-rich fibrin three-dimensional, adhesive, biocompatible and biodegradable scaffold, is obtained.

2. A hyper-acute serum obtained by collecting a blood sample, followed by centrifugation of said blood sample to obtain a platelet-rich fibrin (PRF) clot and an exudate followed by compressing platelet-rich fibrin clot to obtain a membrane and extract the exudate, being the final product hyper-acute serum.

3. A process for the preparation of a platelet-rich fibrin (PRF) three-dimensional, adhesive, biocompatible and biodegradable scaffold and/or membrane and a hyper-acute serum comprising the following steps:
a) collecting blood samples, followed by centrifugation to obtain a platelet-rich fibrin clot and an exudate; and
b) compressing the platelet-rich fibrin clot as obtained in step a) to extract the exudate to obtain the final products being the hyper-acute serum and a platelet-rich fibrin (PRF) three-dimensional, adhesive, biocompatible and biodegradable scaffold and/or membrane.

4. A method for the treatment of inguinal hernia in a patient in need thereof comprising the step of:
a) performing a transverse incision on the inguinal region;
b) positioning a self-retaining retractor with smooth non-traumatic branches into a surgical access created by the incision;
c) preparing with minimal manipulation of the inguinal sac from the spermatic cord and to make a space where a mesh will be located, minimizing detachment of tissues and respecting any nerves that cross the area, while checking hemostasis step by step;
d) isolating and examining contents of the inguinal sac of step c);
e) repositioning the inguinal sac into the patient's abdomen and the internal inguinal ring refashioned using stitches wherein in case of direct hernia the trasversalis fascia is approximated with the same suture material;
f) customizing a mesh to the patient's inguinal region, wherein the mesh is a soft, light, macroporous, monofilament, polypropylene mesh BARD, configured to the shape and size of the inguinal canal and fixed in place with a 2 cm overlap of the mesh above the tubercle;
g) fixing the mesh by using a platelet-rich fibrin three-dimensional, adhesive, biocompatible and biodegradable scaffold and/or membrane and a hyper-acute serum to fix the mesh and secure a tension-free technique prepared by collecting the patient's blood in glass-coated plastic tubes and immediately centrifuging to obtain three distinct layers in the centrifugation tube: red blood corpuscles RBCs at a bottom of the tube, platelet-poor plasma PPP on the top of the tube, and the PRF clot in the middle of the tube, removing the PRF clot was from the tube with surgical tweezers; squeezing out serum from the PRF clot to generates PRF membranes and the hyper-acute serum, PRF clots used to fix the mesh;
h) applying both components, membranes and hyper-acute serum on a posterior fascia (trasversalis) and the mesh attached over them;
i) using two single stitches to adjust the mesh: one in the tubercle area avoiding the periosteum and another one to close the mesh tails;
j) suturing the anterior fascia below the spermatic cord using stitches to press the mesh between the anterior and the trasversalis fascia; and
k) fat suturing and skin intradermic suture.

5. The platelet-rich fibrin (PRF) three-dimensional, adhesive, biocompatible and biodegradable scaffold and/or membrane according to claim 1 obtained by collecting blood samples, followed by centrifugation, wherein the blood sample is 9 ml of fresh blood centrifuged at 2700 rpm for 12 minutes or 10 ml of fresh blood centrifuged at 1500 rpm for 14 minutes.

6. The hyper-acute serum according to claim 2 obtained by collecting blood samples, followed by centrifugation, wherein the blood sample is 9 ml of fresh blood centrifuged at 2700 rpm for 12 minutes or 10 ml of fresh blood centrifuged at 1500 rpm for 14 minutes.

7. The process for the production of a platelet-rich fibrin (PRF) three-dimensional, adhesive, biocompatible and biodegradable scaffold and/or membrane and a hyper-acute serum according to claim 3, wherein the blood sample is 9 ml of fresh blood which is immediately centrifuged at 2700 rpm for 12 minutes or the blood sample is 10 ml of fresh blood immediately centrifuged at 1500 rpm for 14 minutes.

8. The method for the treatment of inguinal hernia according to claim 4, wherein the method is carried out using transabdominal preperitoneal patch plasty (TAPP) laparoscopy, or totally extraperitoneal (TEP) laparoscopy.

9. The method for the treatment of Inguinal Hernia according to claim 4 wherein in step h), three (3) to five (5) L-PRF clots used to fix the mesh.

10. The method for the treatment of inguinal hernia according to claim 4 wherein stitches are polyglactin (vicryl) 2-0 or polyglycolic acid-Vicryl.

11. The method for the treatment of inguinal hernia according to claim 4, wherein the method is carried out under local or general anesthesia.

12. The method for the treatment of inguinal hernia according to claim 4, wherein the transverse incision on the inguinal region is 5-6 cm in length.

13. The method for the treatment of inguinal hernia according to claim 4, wherein the stiches used are polyglactin (vicryl) 2-0 or polyglycolic acid-Vicryl.

14. The method for the treatment of inguinal hernia according to claim 4, wherein the platelet-rich fibrin (PRF) three-dimensional, adhesive, biocompatible and biodegradable scaffold and/or membrane and a hyper-acute serum to fix the mesh and secure a tension-free technique is prepared by collecting 9 ml fresh patient's blood and immediately centrifuging at 2700 rpm for 12 minutes.
